(19) United States
(12) Patent Application Publication
(10) Pub. No.: US 2015/0099940 A1
(43) Pub. Date: Apr. 9, 2015

(54) LUMINAIRE WITH TIR REFLECTOR

(71) Applicant: OSRAM GmbH, Muenchen (DE)

(72) Inventors: Stefan Hadrath, Falkensee (DE); Nico Morgenbrod, Berlin (DE)

(21) Appl. No.: 14/476,770

(22) Filed: Sep. 4, 2014

(30) Foreign Application Priority Data
Oct. 7, 2013 (DE) 102013220218.0

(51) Int. Cl. F21V 7/00; A61B 1/06; F21V 13/02

(52) U.S. Cl. CPC F21V 7/0091; F21V 7/0025; F21V 13/02; A61B 1/06
USPC 600/249; 362/304; 362/235

(57) ABSTRACT

A luminaire includes a reflector arrangement and a light generating unit, which emits its light onto the reflector arrangement. The reflector arrangement includes at least two shell-layer-shaped reflector rings which are arranged coincidentally with regard to their axes of symmetry. The reflector rings have different middle radii, are arranged in a manner one nested in another, and are embodied as total internal reflection reflectors.

### CROSS-REFERENCE TO RELATED APPLICATION

This application claims priority to German Patent Application Serial No. 10 2013 220 218.0, which was filed Oct. 7, 2013, and is incorporated herein by reference in its entirety.

### TECHNICAL FIELD

Various embodiments relate generally to a luminaire, including a reflector arrangement and a light generating unit, which emits its light onto the reflector arrangement, wherein the reflector arrangement is embodied as a TIR reflector. Various embodiments are applicable e.g. to medical luminaires, e.g. surgical luminaires, for vehicle luminaires and for general lighting.

### BACKGROUND

US 2004/0141323 A1 discloses an indicator lamp having an optical axis oriented from the rear side toward the front side, on which axis a light source is provided in order to emit a light flux toward the front and is of the type having an optical device for recovering and distributing rays emitted by the light source, with a view to providing an indicator function complying with regulations, wherein the optical device includes a coaxial ring-shaped reflector and, in front of the light source, a “light engine”, which is provided for distributing the rays of light from the light source in directions that generally run transversely with respect to the optical axis, to be precise in the direction of the coaxial ring-shaped reflector.

WO 2006/043195 A1 discloses a light source having a number of optical components aligned concentrically along an optical axis. The optical components have an arrangement of light emitting diodes, a dielectric collimator having surfaces configured to bring about total internal reflection of light from the arrangement of diodes, and a further reflector in order to further collimate the beam and which can also be based on total internal reflection on account of a prism arrangement on the outer side.

### SUMMARY

A luminaire includes a reflector arrangement and a light generating unit, which emits its light onto the reflector arrangement. The reflector arrangement includes at least two shell-layer-shaped reflector rings which are arranged coincidentally with regard to their axes of symmetry. The reflector rings have different middle radii, are arranged in a manner one nested in another, and are embodied as total internal reflection reflectors.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 shows, as a sectional illustration in side view, a luminaire having two reflector rings in accordance with a first embodiment;

FIG. 2 shows the luminaire in accordance with the first embodiment in a view obliquely from the front;

FIG. 3 shows, as a sectional illustration in side view, the luminaire in accordance with the first embodiment with light paths and an enlarged excerpt from a light generating unit;

FIG. 4 shows, in an oblique view, an excerpt from one of the reflector rings with a possible light path; and

FIG. 5 shows, as a sectional illustration in side view, a luminaire having two reflector rings in accordance with a second embodiment.

### DESCRIPTION

The following detailed description refers to the accompanying drawings that show, by way of illustration, specific details and embodiments in which the invention may be practiced.

The word “exemplary” is used herein to mean “serving as an example, instance, or illustration”. Any embodiment or design described herein as “exemplary” is not necessarily to be construed as preferred or advantageous over other embodiments or designs.

The word “over” used with regards to a deposited material formed “over” a side or surface, may be used herein to mean that the deposited material may be formed “directly on”, e.g. in direct contact with, the implied side or surface. The word “over” used with regards to a deposited material formed “over” a side or surface, may be used herein to mean that the deposited material may be formed “indirectly on” the implied side or surface with one or more additional layers being arranged between the implied side or surface and the deposited material.

Various embodiments may at least partly overcome the disadvantages of the prior art.

Various embodiments provide a luminaire, including a reflector arrangement and a light generating unit, which emits its light onto the reflector arrangement. The reflector arrangement includes at least two shell-layer-shaped reflector rings which are arranged coincidentally with regard to their axes of symmetry, which reflector rings have different middle radii, are arranged in a manner one nested in another and are embodied as TIR (total internal reflection) reflectors.

The use of a plurality of reflector rings makes it possible to realize even complex and/or highly focusing emission patterns with a comparatively low outlay. In various embodiments, the beam shaping angles or aperture angles of the light beams emitted by the reflector rings can be kept particularly small, which brings about an advantageous height insensitivity of the light emission pattern. The nested or interleaved arrangement affords the additional advantage that the reflector arrangement and thus the luminaire can have a particularly flat design which, with a single, continuous reflector, cannot be provided or can be provided only in a manner involving very high outlay.

In one development, a beam shaping angle of a light beam of at least one of the reflector rings is less than 20°, e.g. less than 18°, in particular less than 15°, e.g. less than 10°, e.g. less than 7.5°. In various embodiments, the beam shaping angles of the light beams of all the reflector rings may lie within these limits. The beam shaping angles of the light beams of different reflector rings may be identical or different. A superimposition of the light beams may be achieved in a predetermined target plane or in the far field.

A shell-layer-shaped reflector ring is understood to mean, for example, a ring-shaped reflector whose basic shape arises in an imaginary manner as a result of a layer-like section cut from an in particular thin-walled hollow body. The layer-like section may be e.g. a section cut transversely with respect to an axis of symmetry or a longitudinal axis of the imaginary hollow body.

A reflector ring may be e.g. a radially widening body having a smaller opening (designated as “ring opening” hereinafter, without restricting the generality) and a larger ring opening. The smaller ring opening is delimited by the narrower edge of the reflector ring and the larger ring opening is delimited by the further edge of the reflector ring. The ring openings or edges may be circular, but—depending on the shape of the imaginary hollow body—are not restricted thereto and may alternatively be e.g. oval or even angular.

The imaginary hollow body may be e.g. a hollow body of revolution, e.g. a spherical shell, a paraboloid of revolution, etc., but is not restricted thereto. In this regard, e.g. a front side facing the light generating unit, on which front side the light emitted by the light generating unit thus impinges, may have a circle-like, oval, parabolic, but also freeform, etc., cross-sectional shape. The reflector rings may e.g. be spaced apart from one another, e.g. by gaps or by non-illuminated regions of the reflector arrangement.

A radius is understood to mean e.g. a radius perpendicular to the associated axis of symmetry. A middle radius is understood to mean e.g. a radius at middle height of a reflector ring along its axis of symmetry and at middle width perpendicular to its axis of symmetry.

At least two different reflector rings may have the same focal point or focal spot. Alternatively or additionally, two different reflector rings may have a different focal point or focal spot.

The reflector rings can be constructed integrally or in a multipartite manner. In the case of a multipartite construction, small gaps between adjacent individual parts may be harmless in practice.

The fact that two reflector rings are arranged in a manner nested one in another encompasses e.g. the fact that they at least partly intersect or overlap along their common axis of symmetry. In one configuration, for example, two adjacent reflector rings at least partly intersect or overlap along the axis of symmetry. In the intersection region, the reflector ring having the larger middle radius surrounds e.g. the reflector ring having the smaller middle radius.

In one development, a position of the reflector rings along the axis of symmetry is fixed. Alternatively, a position along the axis of symmetry may be adjustable, e.g. adjustable by the user, in particular the distance between reflector rings and/or from the reflector rings to the light generating unit. As a result, the light emission pattern can be adapted in a simple manner.

The fact that a reflector ring is embodied as a TIR reflector encompasses e.g. the fact that its reflectivity is at least partly based on total internal reflection, that is to say that the reflector ring is a TIR body. In one development, the reflector ring partly has a reflective coating. In one preferred development, the reflector ring does not have a reflective coating for producing its reflection capability, but rather produces the latter just on account of its property as a TIR body.

The reflector rings as TIR reflectors includes or essentially consist e.g. of a transparent material, for example of transparent plastic (such as PMMA, PC, ABS, etc.) or of glass.

The light generating unit may emit its light at the focal point or focal spot of at least one of the reflector rings. The light generating unit may alternatively or additionally emit its light outside a focal point or focal spot of at least one of the reflector rings, e.g. in a manner slightly offset therefrom, e.g. offset on the axis of symmetry.

The light generating unit may be embodied e.g. as a module or light generator (“light engine”) and e.g. be exchangeable, e.g. even by the user or by a service engineer. As a result, a light emission pattern of the luminaire may be varied using simple means, e.g. with regard to its luminous flux, its color, etc.

In one configuration, the reflector rings, along their axis of symmetry, are further away from the light generating unit, the smaller their middle radius.

In another configuration, adjacent reflector rings are spaced apart from one another in a manner that is gapped radially with respect to the axis of symmetry. In other words, there is at least one gap between the two adjacent reflector rings. Said gap enables air to pass through the reflector arrangement. This may improve cooling of the reflector arrangement, may prevent a beam quality-reducing shimmer of hot air between the light generating unit and the reflector rings and may improve an air flow behavior in the surrounding space.

In one configuration, adjacent reflector rings are spaced apart from one another in a manner that is gapped radially with respect to the axis of symmetry. The gap is therefore also present upon consideration along the axis of symmetry, which enables a particularly effective passage of air.

In one development which may be provided for air circulation, adjacent reflector rings are spaced apart from one another by a ring gap or by a ring composed of ring sectors or ring segments spaced apart from one another. Providing a ring gap may afford the advantage that the reflector rings can be produced separately using simple means. Connecting webs possibly present which bridge the ring gap and serve for connecting the two reflector rings may be able to be disregarded in practice for a through-flow. A through-flow of air through such a reflector arrangement may still take place through the central opening thereof. The central opening may correspond for example to the smaller ring opening of the reflector ring having the smallest middle radius.

In a further configuration, adjacent reflector rings are connected to one another in a closed manner, that is to say have no gap between them. This enables simple integral production in a single work step, e.g. by means of an injection molding. Moreover, in this way the positioning of the two reflector rings can be set particularly fixedly and accurately. Connecting regions present between adjacent reflector rings are not irradiated by light from the light generating unit.

In one configuration which may be provided for a high reflectivity in conjunction with low light losses, the reflector rings include or essentially consist of transparent material, the light from the light generating unit is incident at the front side of said reflector rings and the rear side of said reflector rings is designed for total internal reflection of the incident light.

In one configuration thereof, the reflector rings have a tooth-like or toothed TIR structure at their rear side. A tooth-like structure, e.g. similar to a gearwheel, can be realized in a simple manner and is effective. The teeth are shaped e.g. in a profile-like manner.

In one configuration, furthermore, the toothed TIR structure has a plurality of triangular ribs aligned in a meridian-like manner and arranged alongside one another in the circumferential direction. This can be realized in a particularly simple manner and is effective. A triangular rib may be understood to mean in particular an elongate, profile-like region having a triangular cross-sectional area. A ray of light reflected there by means of total internal reflection may be reflected in particular twice in the ribs. However, ribs having any other suitable cross-sectional shape may also be used. Generally, the TIR structure may also be embodied differently, e.g. in the form of pads, rings, etc. A “meridian-like” alignment may be understood to mean e.g. an alignment along the meridians of the associated imaginary hollow body.

From a different standpoint, the TIR structure may have grooves or slots which can e.g. also be regarded as interspaces between ribs. Said grooves may likewise be V-shaped or triangular and may directly adjoin one another.

In one configuration, moreover, the light generating unit includes one or a plurality of semiconductor light sources for generating light. In various embodiments, the at least one semiconductor light source includes at least one light emitting diode. If a plurality of light emitting diodes are present, they can emit light in the same color or in different colors. A color can be monochromatic (e.g. red, green, blue, etc.) or multichromatic (e.g. white). Moreover, the light emitted by the at least one light emitting diode can be an infrared light (IR LED) or an ultraviolet light (UV LED). A plurality of light emitting diodes can generate a mixed light; e.g. a white mixed light. The at least one light emitting diode can contain at least one wavelength-converting phosphor (conversion LED). Alternatively or additionally, the phosphor can be arranged in a manner remote from the light emitting diode (“remote phosphor”). The at least one light emitting diode can be present in the form of at least one individually packaged light emitting diode or in the form of at least one LED chip. A plurality of LED chips can be mounted on a common substrate (“submount”). The at least one light emitting diode can be equipped with at least one dedicated and/or common optical unit for beam guiding, e.g. at least one Fresnel lens, collimator, etc. Instead of or in addition to inorganic light emitting diodes, e.g. based on InGaN or AlInGaP, generally organic LEDs (OLEDs, e.g. polymer OLEDs) can also be used. Alternatively, the at least one semiconductor light source may include e.g. at least one diode laser. The latter may be used e.g. together with remote phosphor, e.g. in the sense of a so-called LARP (“Laser Activated Remote Phosphor”) concept.

In one configuration, furthermore, the light generating unit includes at least one collimator which is disposed downstream of the at least one semiconductor light source and the light from which is incident on a reflector (designated as “primary reflector” hereinafter, without restricting the generality) for deflection onto the reflector arrangement. For this purpose, the primary reflector may be reflectively coated. The primary reflector may be designed in the sense of a remote phosphor body to carry out at least partial wavelength conversion of the light incident on it. If the semiconductor light sources are lasers, the primary reflector may then also be designated as a LARP reflector.

In one configuration, in addition, the luminaire is a medical luminaire, e.g. surgical luminaire. The luminaire is particularly suited thereto since it is compact and enables even complex light emission patterns with high quality, e.g. without shimmer on account of air heating. Moreover, what is particularly appreciated in this case is that an air flow behavior in the surrounding space is impeded to a comparatively small extent.

However, the luminaire is not restricted thereto, but rather may e.g. also serve as a vehicle luminaire, e.g. as a headlight or as an indicating luminaire such as travel direction indicator, brake light, etc. Moreover, the luminaire may advantageously be used as a general point emitter or spot, e.g. for general lighting indoors (e.g. for room lighting, e.g. in desk lamps or uplighters) or outdoors (e.g. for street lighting or object lighting).

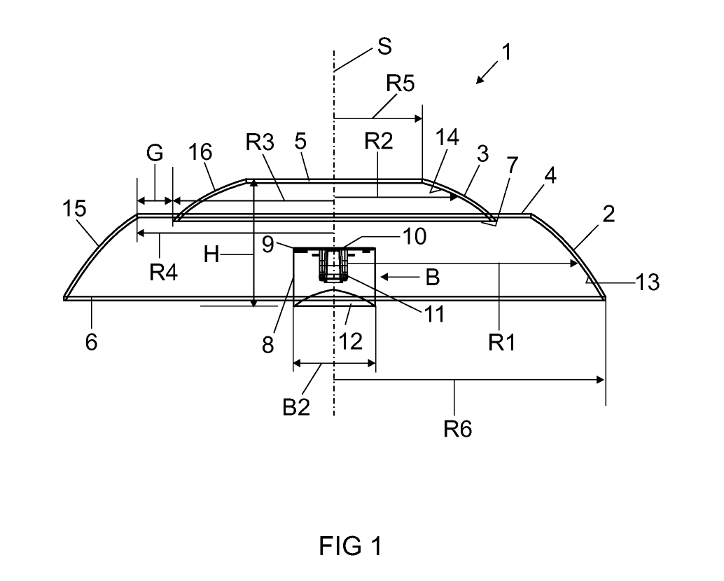

FIG. 1 shows a luminaire for medical purposes in the form of a surgical luminaire 1, including a reflector arrangement 2, 3 constructed from two shell-layer-shaped reflector rings 2 and 3. The reflector rings 2 and 3 have a basic shape here in accordance with a parabolic shell layer or paraboloid layer. Their axes S of symmetry are coincident or congruent. The reflector rings 2 and 3 open in the same direction along the axis S of symmetry. The reflector rings 2 and 3 consist of transparent material, e.g. of glass or plastic, e.g. PMMA, polycarbonate.

A first, larger reflector ring 2 of the reflector rings 2 and 3 has a larger middle radius R1 with respect to the axis S of symmetry, while a second, smaller reflector ring 3 has a smaller middle radius R2 with respect to the axis S of symmetry. Each of the reflector rings 2 and 3, on account of its shape that widens continuously along the axis of symmetry, has a small ring opening 4 and 5, respectively, and a large ring opening 6 and 7, respectively.

In a focal region or focal spot B of the reflector rings 2 and 3 or in a manner slightly offset therefrom, a light generating unit 8 radiates its light onto the reflector rings 2 and 3. The focal spot B may have a certain extent, which, however, in practice does not impair a directional effect of the reflector rings 2 and 3. The light generating unit 8 includes a plurality of semiconductor light sources in the form of LEDs 10 fitted on a common substrate 9. A tubular collimator 11 is disposed downstream of the LEDs 10 and concentrates or collimates the light emitted by the LEDs 10 along the axis S of symmetry. The collimated light is incident on a primary reflector 12 which reflects the light onto the reflector rings 2 and 3.

The reflector rings 2 and 3 are interleaved one in another in such a way that they partly overlap along the common axis S of symmetry. In this case, the smaller reflector ring 3 is further away from the light generating unit 8 than the larger reflector ring 2 along the axis S of symmetry. The light generating unit 8 is surrounded by the larger reflector ring 2 almost over its entire height along the axis S of symmetry. This interleaved arrangement produces a particularly compact reflector arrangement 2, 3.

A maximum radius R3, which determines the largest radial extent of the smaller reflector ring 3, at the large ring opening 7 thereof is smaller than a minimum radius R4 at the small ring opening 4 of the larger reflector ring 2. As a result, the smaller reflector ring 3 can dip freely into the larger reflector ring 2, and a ring gap G having the width R4-R3 between the two reflector rings 2 and 3 arises in a view along the axis S of symmetry. The reflector rings 2 and 3 are therefore spaced apart from one another in a manner that is gapped radially with respect to the axis S of symmetry. The ring gap G enables an airflow between the reflector rings 2 and 3, which improves the cooling thereof and reduces beam quality-reducing air turbulence and air accumulation of warm air.

It is generally provided if the maximum radius R3 of the smaller reflector ring 3 is in a range of between 180 mm and 220 mm, e.g. approximately 200 mm. Furthermore, it is generally provided if a minimum radius R5 at the small ring opening 5 thereof is in a range of between 90 mm and 130 mm, e.g. approximately 110 mm. It is additionally generally provided if the minimum radius R4 of the larger reflector ring 2 at the small ring opening 4 thereof is in a range of between 230 mm and 270 mm, e.g. approximately 250 mm. It is additionally generally provided if the maximum radius R6 of the larger reflector ring 2 is in a range of between 320 mm and 360 mm, e.g. approximately 340 mm. The width of the ring gap G may then e.g. be between 40 mm and 60 mm, e.g. approximately 50 mm. A height H of the luminaire 1 along the axis S of symmetry having both reflector rings 2 and 3 and the light generating unit 8 may be between 140 mm and 180 mm, e.g. approximately 160 mm. A height of the light generating unit 8 may be between 65 mm and 85 mm, e.g. approximately 85 mm. A width B2 of the light generating unit 8 may be between 90 mm and 110 mm, e.g. approximately 100 mm.

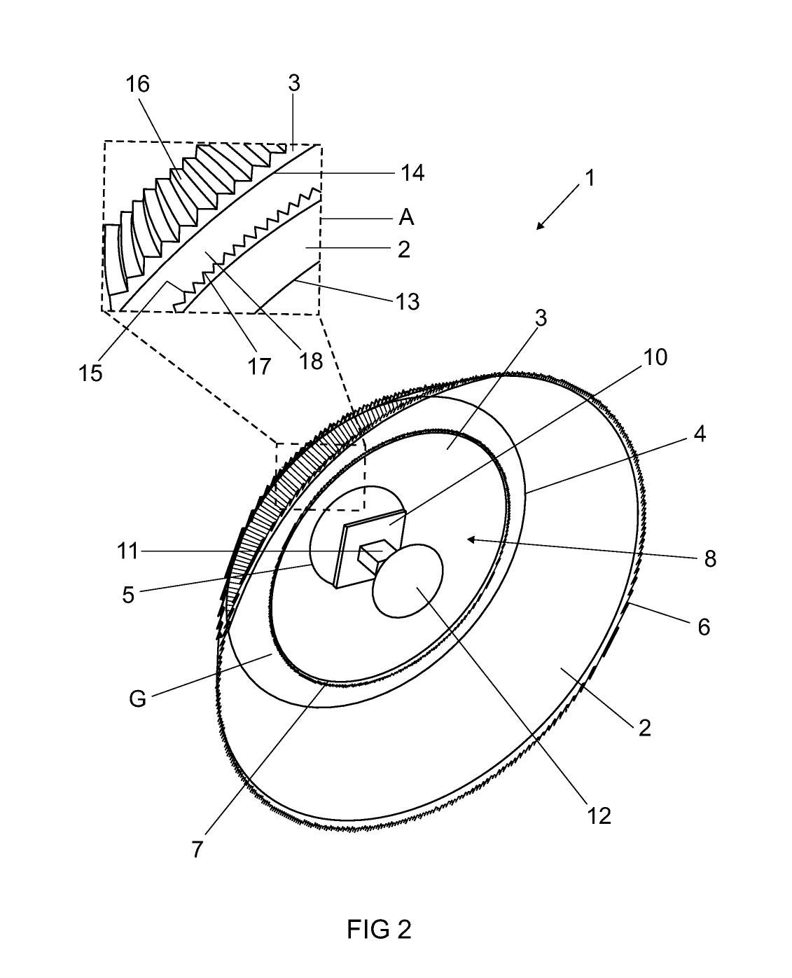

FIG. 2 shows the surgical luminaire 1 in a view obliquely from the front. As shown in an enlarged excerpt A, the front sides 13 and 14 of the reflector rings 2 and 3, respectively, on which the light emitted by the primary reflector 12 is incident, are embodied in a smooth fashion. By contrast, the corresponding rear sides 15 and 16 have a toothed structure composed of ribs 17 and grooves 18, respectively, aligned in a meridian-like manner and arranged alongside one another in the circumferential direction. The ribs 17 and grooves 18 have a V-shaped or triangular cross section. As a result, the rear sides 15 and 16 are embodied as TIR structures suitable for total internal reflection. The reflector rings 2 and 3 are therefore TIR reflectors which, for example, require no reflective coating (metalization or the like).

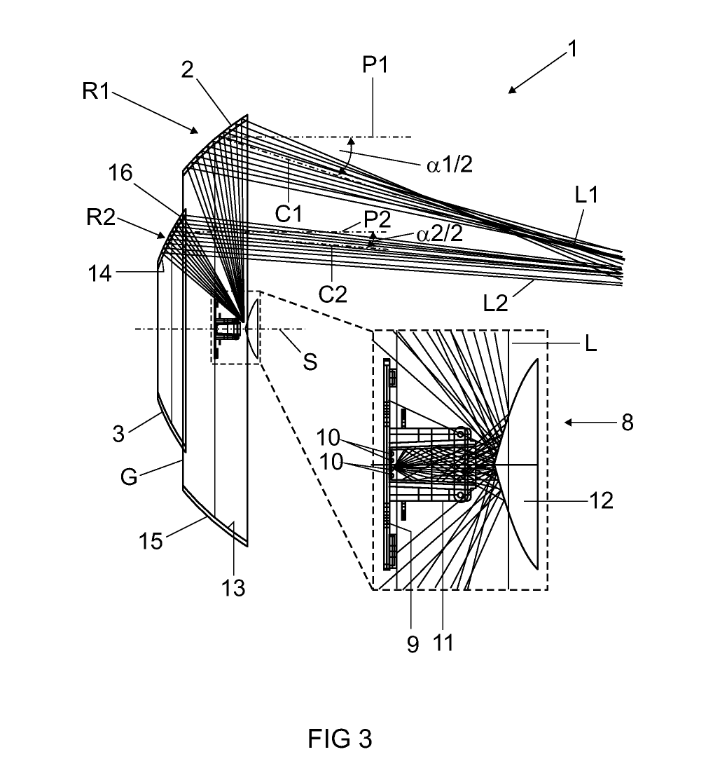

As shown in greater detail in FIG. 3, light L is emitted by the LEDs 10 during the operation of the light generating unit 8, said light being concentrated or collimated in a collimator 11 in the form of a slightly widening small tube embodied in an internally reflecting manner. A longitudinal axis of the collimator 11 here lies on the axis S of symmetry, for example. The primary reflector 12 disposed downstream of the collimator 11 is positioned at a distance in front of the collimator 11 and reflects the light L emitted by the collimator 11 onto the two reflector rings 2 and 3.

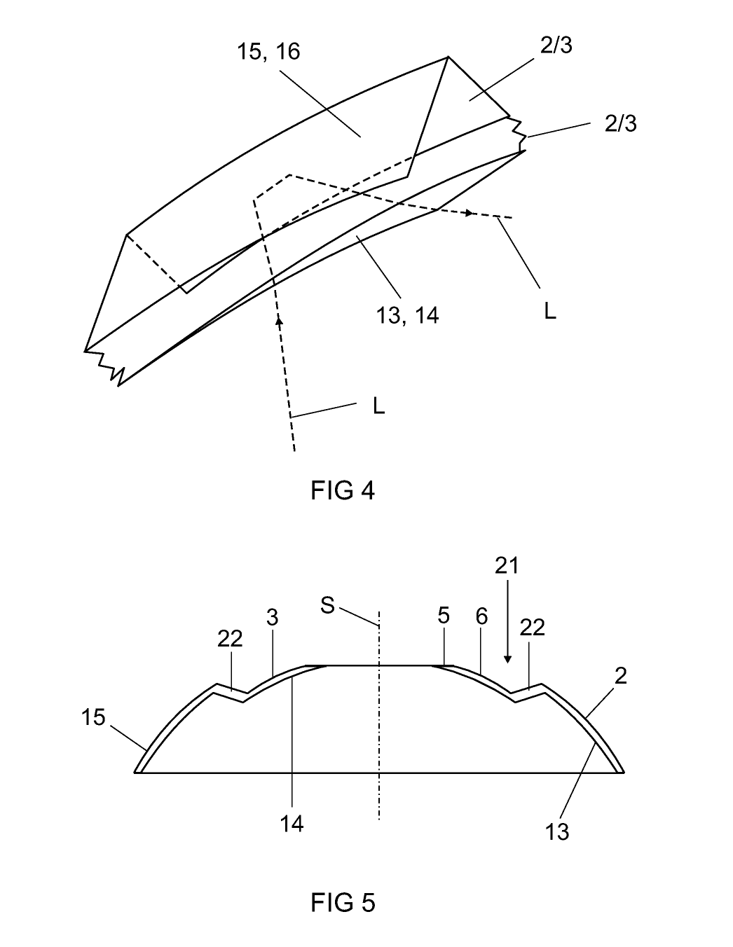

As also shown in greater detail as an excerpt in an oblique view in FIG. 4, light L impinging on and then entering the reflector rings 2 and 3 passes through the reflector rings 2 and 3 until it reaches the respective rear sides 15 and 16, where, as shown, it is internally reflected twice at the ribs 17 and is then reflected back onto the front sides 13 and 14, respectively.

Referring to FIG. 3 again, respective light beams L1 and L2 are ultimately generated by the two reflector rings 2 and 3. The light beams L1, L2 may be directed e.g. in different directions, e.g. focused differently. The light beams L1, L2 may have e.g. a beam shaping angle of less than 20°, wherein here e.g. the light beam L2 may have a smaller beam shaping angle than the light beam L1. A superimposition of the light beams L1, L2 may be achieved in a predetermined target plane (e.g. on a patient) or in the far field.

FIG. 3 depicts, to put it more precisely, a first half beam shaping angle α½ of the light beam L1. This half beam shaping angle α½ relates to an angle at the middle radius R1 of the first reflector ring 2 between a line P1 parallel to the axis S of symmetry and a first light ray C1 proceeding from the middle radius R1. A second half beam shaping angle α2/2 of the light beam L2 is depicted analogously. This half beam shaping angle α2/2 analogously relates to an angle at the middle radius R2 of the second reflector ring 3 between a line P2 parallel to the axis S of symmetry and a second light ray C2 proceeding from the middle radius R2. The first half beam shaping angle α½ of the light beam L1 here may be e.g. between 8° and 10°, e.g. approximately 8.5°. The second half beam shaping angle α2/2 of the light beam L2 is half of that, that is to say e.g. between 4° and 5°, e.g. approximately 4.25°. In one general configuration, the beam shaping angle α2 of a smaller reflector ring 3 is smaller than the beam shaping angle α1 of a larger reflector ring 3, and e.g. assumes only half the value.

Generally, the reflector rings 2 and 3 may have identical or different focal regions or focal spots, e.g. offset with respect to one another along the axis S of symmetry.

FIG. 5 shows a reflector arrangement 21 comprising two reflector rings 2 and 3, which are now connected to one another in a closed manner or without a gap and thus form reflector regions of an integral closed reflector arrangement 21. Consequently, air cannot flow through between the two reflector rings 2 and 3, but rather only through the small ring opening 5 of the smaller reflector ring 3. The connecting region 22 that connects the reflector rings 2 and 3 to one another is not irradiated by light.

Although the light beams emitted by the reflector rings are shown as convergent or converging light beams in the figures, generally divergent or diverging light beams may also be generated.

Although two reflector rings are shown in the figures, generally more than two reflector rings may also be used, e.g. 3, 4 or even more reflector rings.

Generally, “a”, “one”, etc. can be understood to mean a singular or a plural, in particular in the sense of “at least one” or “one or a plurality”, etc., as long as this is not explicitly excluded, e.g. by the expression “exactly one”, etc.

Moreover, a numerical indication can encompass exactly the indicated number and also a customary tolerance range, as long as this is not explicitly excluded.

### LIST OF REFERENCE SIGNS

1 surgical luminaire
2 larger reflector ring
3 smaller reflector ring
4 small ring opening of the larger reflector ring
5 small ring opening of the smaller reflector ring
6 large ring opening of the larger reflector ring
7 large ring opening of the smaller reflector ring
8 light generating unit
9 substrate
10 LED
11 collimator
12 primary reflector
13 front side of larger reflector ring
14 front side of smaller reflector ring
15 rear side of larger reflector ring
16 rear side of smaller reflector ring
17 rib
18 groove
21 reflector arrangement
22 connecting region
A excerpt
α1 beam shaping angle of the larger reflector ring
α2 beam shaping angle of the smaller reflector ring
B focal region/focal spot
C1 light ray proceeding from a middle radius R1
C2 light ray proceeding from a middle radius R2
G ring gap
H height of the luminaire
L light
L1 light beam
L2 light beam
P1 line parallel to the axis of symmetry at the middle radius R1
P2 line parallel to the axis of symmetry at the middle radius R2
R1 middle radius of the larger reflector ring
R2 middle radius of the smaller reflector ring
R3 maximum radius of the smaller reflector ring
R4 minimum radius of the larger reflector ring
R5 minimum radius of the smaller reflector ring
R6 maximum radius of the larger reflector ring
S axis of symmetry

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

What is claimed is:

1. A luminaire, comprising:
a reflector arrangement; and
a light generating unit, which emits its light onto the reflector arrangement;
wherein the reflector arrangement comprises at least two shell-layer-shaped reflector rings which are arranged coincidentally with regard to their axes of symmetry;
wherein the reflector rings have different middle radii, are arranged in a manner one nested in another, and are embodied as total internal reflection reflectors.

2. The luminaire of claim 1,
wherein a beam shaping angle of a light beam of at least one of the reflector rings is less than 20°.

3. The luminaire of claim 2,
wherein a beam shaping angle of a light beam of all the reflector rings is less than 20°.

4. The luminaire of claim 1,
wherein the reflector rings, along their axis of symmetry, are further away from the light generating unit, the smaller their middle radius.

5. The luminaire of claim 1,
wherein two adjacent reflector rings at least partly overlap along their axis of symmetry.

6. The luminaire of claim 1,
wherein adjacent reflector rings are spaced apart from one another in a manner that is gapped radially with respect to their axis of symmetry.

7. The luminaire of claim 1,
wherein adjacent reflector rings are connected to one another in a closed manner.

8. The luminaire of claim 1,
wherein the reflector rings comprise or essentially consist of transparent material, the light from the light generating unit is incident at the front side of said reflector rings and the rear side of said reflector rings is designed for total internal reflection of the incident light.

9. The luminaire of claim 1,
wherein the reflector rings have a toothed total internal reflection structure at their rear side.

10. The luminaire of claim 9,
wherein the toothed total internal reflection structure has a plurality of triangular ribs aligned in a meridian-like manner and arranged alongside one another in the circumferential direction.

11. The luminaire of claim 1,
wherein the light generating unit comprises one or a plurality of semiconductor light sources for generating light.

12. The luminaire of claim 11,
wherein the light generating unit comprises at least one collimator which is disposed downstream of the at least one semiconductor light source and the light from which is incident on a primary reflector for deflection onto the reflector arrangement.

13. The luminaire of claim 1,
wherein the luminaire is a medical luminaire.

14. The luminaire of claim 13,
wherein the medical luminaire is a surgical luminaire.
